(19) United States
(12) Patent Application Publication
(10) Pub. No.: US 2021/0349136 A1
(43) Pub. Date: Nov. 11, 2021

(54) EXAMINING OBJECTS USING ELECTRIC FIELDS

(71) Applicant: Zedsen Limited, London (GB)

(72) Inventor: Hrand Mami Mamigonians, London (GB)

(21) Appl. No.: 17/314,249

(22) Filed: May 7, 2021

(30) Foreign Application Priority Data
May 7, 2020 (EP) 20173579.2

(51) Int. Cl. G01R 27/26; G01N 27/22

(52) U.S. Cl. CPC G01R 27/2605; G01N 27/221

(57) ABSTRACT

Objects are examined, including biological objects, possibly to determine concentrations of constituents in flowing blood. An input circuit supplies an input signal to a transmitter electrode and an output signal receives an output signal from a receiver electrode, wherein the transmitter electrode and the receiver electrode are capacitively coupled. A variable resistance circuit is connected in series between an input circuit and a transmitter electrode. The input signal is an impulse signal with an initial edge having a transition time. This transition time is adjustable in response to varying the resistance presented by the variable resistance circuit.

### CROSS REFERENCE TO RELATED APPLICATIONS

This application claims priority from European Patent Application number EP 20 173 579.2, filed on May 7, 2020, the whole contents of which are incorporated herein by reference.

### BACKGROUND OF THE INVENTION

The present invention relates to an apparatus for examining objects, of the type comprising at least one transmitter electrode and at least one receiver electrode; an input circuit for supplying an input signal to said at last one transmitter electrode; and an output circuit for receiving an output signal from said at least one receiver electrode, wherein said transmitter electrode and said receiver electrode are capacitively coupled.

The present invention also relates to a method of examining objects, of the type in which an electric field passes through an object in response to energizing a transmitter electrode and monitoring a receiver electrode.

It is known to use electric fields to determine the composition of materials. For example, as disclosed in US2019/086352A1, assigned to the present applicant, it is possible to use electric fields to scan shoes to identify hidden materials; typically, prior to boarding public transportation. To achieve the required level of penetration, relatively high input voltages are deployed (typically ranging from ten volts to over three hundred volts) which in turn places constraints on the extent to which signals of this type may be modified.

It has also been shown, using signals of lower intensity, that a similar approach may be adopted to identify blood constituents, such as glucose. The applicant has identified that a demand exists for the production of monitors of this type that can be deployed in a substantially mobile environment. Standalone devices have been proposed but it has also been appreciated that advantages can be gained by facilitating communications with mobile communication devices, such as mobile cellular telephones. Development work in this area has also identified opportunities for making changes to the shape of input impulse signals, given that operation within a mobile environment of this type may take place using substantially lower voltages (typically, below five volts). A problem therefore exists in terms of how to introduce appropriate modifications to facilitate the measurement of concentrations of particular blood constituents, or other material in other environments, using sophisticated circuitry that could not be deployed in the higher voltage systems.

### BRIEF SUMMARY OF THE INVENTION

According to a first aspect of the present invention, there is provided an apparatus for examining objects, comprising: at least one transmitter electrode and at least one receiver electrode; an input circuit configured to supply an input signal to said at least one transmitter electrode; an output circuit arranged to receive an output signal from said at least one receiver electrode, wherein said transmitter electrode and said receiver electrode are capacitively coupled; a variable resistance circuit connected in series between said input circuit and said transmitter electrode, wherein: said input signal is an impulse signal with an initial edge having a transition time; and said transition time is adjustable in response to varying the resistance presented by said variable resistance circuit.

The objects being examined may include living tissue, biological specimens or non-biological objects. In response to an input signal, the output signal will vary due to electrical characteristics of the object which in turn reflects the material composition of the object. When examining living tissue, an electric field may pass through circulating blood, thereby allowing an assessment to be made of the concentration of a constituent in the blood. This constituent could be biological, such as a protein; organic, such as glucose, or inorganic.

Research has shown that the electrical properties of objects and substances, and in particular their permittivity and conductivity, are sensitive to the frequency of applied input signals. Experimental systems have been developed that perform tests by applying many signals of differing frequencies at the same location. A problem with this approach is that the equipment is expensive, time consuming and energy demanding. Consequently, such systems are not appropriate for portable mass deployment.

As an alternative to deploying many input signals for which each has a specific frequency, frequency-dependent data can be derived by deploying non-sinusoidal input signals of differing shapes, which may be seen as being built up from many frequency components. The present invention deploys an impulse, with a sharp rising or falling transition. A transition of this type cannot be instantaneous and higher frequency components are present as the sharpness of the transition increases; that is to say, the transition time decreases.

Experiments have shown that some frequencies are more sensitive that others at detecting the presence of particular constituents. Furthermore, the inventor has appreciated that it is possible to increase the sensitivity of a sensor be adjusting the extent to which high frequency components are present within an input signal. Thus, in response to identifying the particular constituent of interest, such as glucose within circulating blood, it is possible to optimize the frequency components of an input energizing circuit. Furthermore, when applying an impulse, frequency components can be adjusted by adjusting the transition time.

The relationship between the transmitter electrode and the receiver electrode is that of them being capacitively coupled. This coupling is achieved due to capacitance introduced by the permittivity of the object under examination. The size of the capacitance will influence the rate at which a transmission occurs, along with any resistance included within the energizing lines. The capacitance of the object cannot be modified because it is the variations in permittivity and conductivity of the object that allow a determination of its constituents to be determined. However, the invention proposes to control a series-connected resistance, which in turn will change the rate of transition, which in turn will adjust the individual frequency components of the input impulse signal.

In an embodiment, the variable resistance circuit comprises a plurality of resistors that are selectively connected between the input circuit and the transmitter electrode. In alternative embodiments, other resistive elements could be deployed and resistance could be established by active circuits.

In an embodiment, the apparatus further comprises a multiplexing device for selecting one of the plurality of resistors. Alternatively, other switching systems could be deployed.

According to a second aspect of the present invention, there is provided a method of examining objects, comprising the steps of: passing an electric field through an object, in response to energizing a transmitter electrode; and monitoring a receiver electrode, wherein: an energizing signal is an impulse signal with an initial edge having a transition time; and said transition time is adjusted to optimize response characteristics of the examination with respect to particular components of interest.

Embodiments of the invention will be described, by way of example only, with reference to the accompanying drawings. The detailed embodiments show the best mode known to the inventor and provide support for the invention as claimed. However, they are only exemplary and should not be used to interpret or limit the scope of the claims. Their purpose is to provide a teaching to those skilled in the art. Components and processes distinguished by ordinal phrases such as “first” and “second” do not necessarily define an order or ranking of any sort.

### BRIEF DESCRIPTION OF THE SEVERAL VIEWS OF THE DRAWINGS

FIG. 1 shows an apparatus for examining objects;

FIG. 2 details a touch sensitive visual display of a mobile cellular telephone;

FIG. 3 illustrates methods performed using the apparatus of FIG. 1;

FIG. 4 shows a schematic representation of the apparatus of FIG. 1;

FIG. 5 shows the energizing circuit identified in FIG. 4;

FIG. 6 shows examples of impulse signals;

FIG. 7 shows a representation of frequency components;

FIG. 8 shows an example of a multiplexing environment identified in

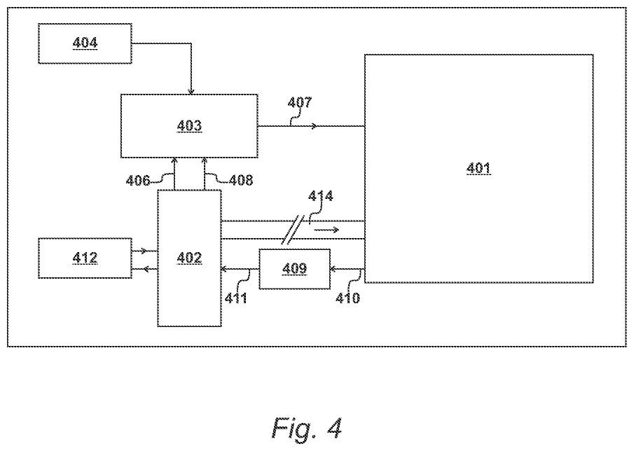

FIG. 4;

FIG. 9 shows the provision of two mutually orthogonal electrode groups;

FIG. 10 shows the analog processing circuit identified in FIG. 4;

FIG. 11 illustrates electric fields generated by the first group of electrodes; and

FIG. 12 illustrates the sampling of an output signal.

### DETAILED DESCRIPTION OF EMBODIMENTS OF THE INVENTION

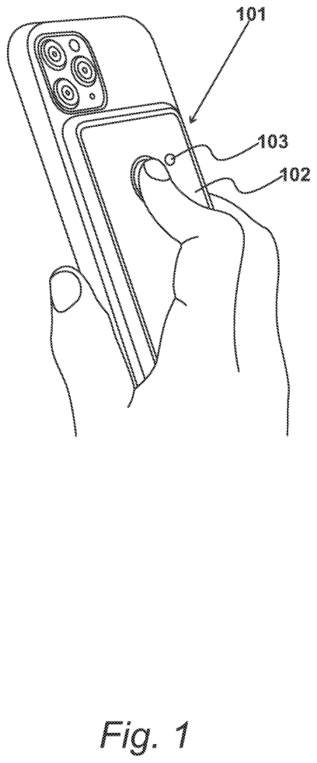

FIG. 1

An apparatus 101 for examining objects is shown in FIG. 1, having at least one transmitter electrode and at least one receiver electrode. An input circuit supplies an input signal to the at least one transmitter electrode and an output circuit receives an output signal from the at least one receiver electrode, wherein the transmitter electrode and the receiver electrode are capacitively coupled.

Many arrangements of electrodes are possible however, in an embodiment, the at least one transmitter electrode and the at least one receiver electrode are selected from a first set of substantially parallel electrodes.

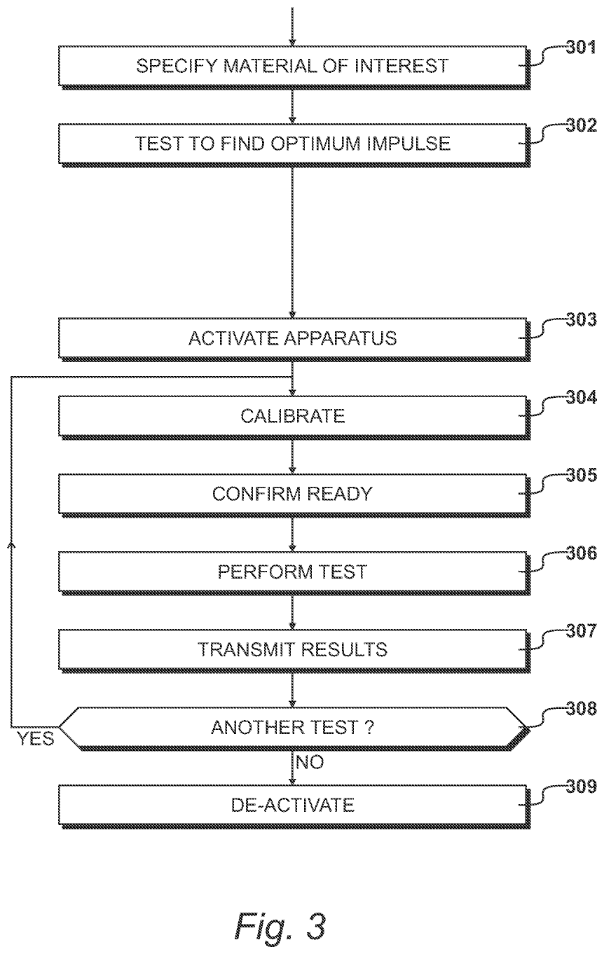

The features described herein, to achieve modifications to input impulses, may be deployed advantageously in many examining environments. In the embodiment shown in FIG. 1, a housing 102 facilitates an interaction with a finger upon the first set of substantially parallel electrodes to facilitate the measurement of a constituent within the blood. The adaptations described herein facilitate optimization operations to enhance the sensitivity to particular components of the blood, given that these behave differently in response to applied fields. In particular, much work has been done in terms of deploying sinusoidal waves of differing frequencies, requiring a frequency sweep to be performed. The present embodiment seeks to ensure that, within an impulse signal, appropriate frequencies are present for particular blood constituents. In the embodiment shown, a preprograming operation has been performed, described further with reference to FIG. 3, to optimize the device for the detection of glucose levels within the blood.

An embodiment also includes devices for measuring applied pressure, to ensure that an appropriate pressure has been applied. The present embodiment also includes a temperature measuring device 103. The temperature measuring device may make use of an infra-red sensor that can detect the temperature of an applied finger without being in physical contact with the finger.

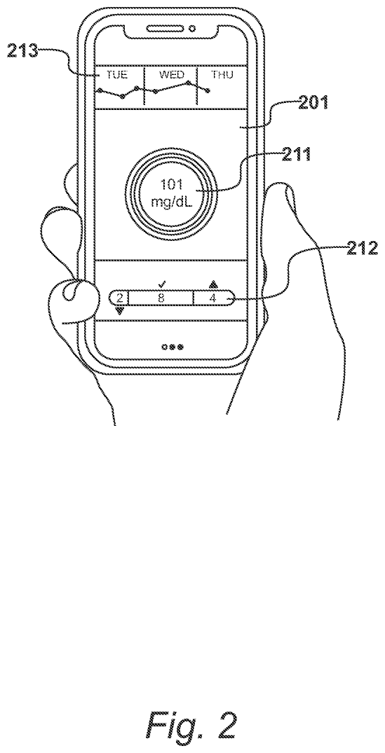

FIG. 2

In this embodiment, a touch sensitive visual display 201 of a mobile cellular telephone is used to facilitate a subject's interaction with the apparatus. An appropriate software application is installed upon the mobile telephone, thereby allowing a subject to initiate a glucose measuring episode. Graphical areas then allow information to be displayed.

In the graphical user interface illustrated in FIG. 2, a first element 211 shows the results of a current measurement taken, as described with reference to FIG. 1. A second element 212 indicates whether the measurement is considered low, normal or high. This in turn prompts the subject to make the appropriate medical intervention. Furthermore, a third element 213 displays historical data, thereby allowing trends to be considered.

FIG. 3

The apparatus described with reference to FIG. 1 and FIG. 2 may be used to consider biological, organic and non-organic substances circulating within the blood. Furthermore, techniques described herein may also be deployed in other environments for detecting constituents within sample tissues or in non-biological material. Consequently, as shown in FIG. 3, a first step 301 involves specifying the material of interest. In an embodiment, the material of interest is glucose of the type found in circulating blood but other blood circulating substances could be specified, such as sodium.

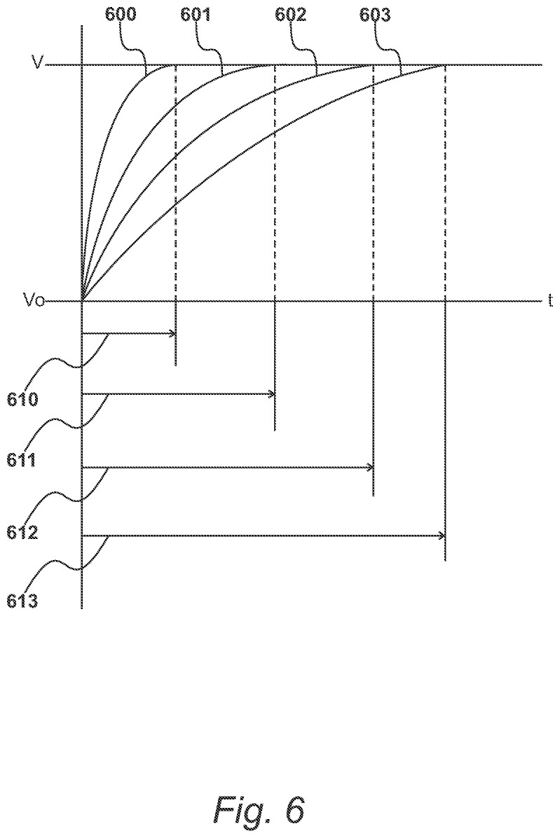

Having specified the material of interest at step 301, a test is performed to find an optimum impulse shape, from the impulse shapes that can be produced by the system. Thus, an electric field passes through an object in response to energizing a transmitter electrode and monitoring a receiver electrode. The energizing signal is an impulse signal with an initial edge having a transition time. For the purposes of illustration, a rising edge is considered, as described with reference to FIG. 6, but it can be appreciated that from a polarity perspective, a falling edge could achieve substantially similar results.

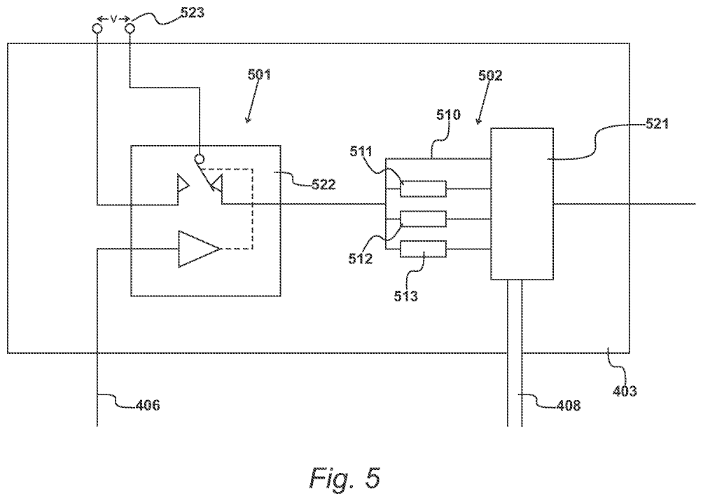

As known to those skilled in the art, an impulse signal may be considered as being constructed from a range of sinusoidal frequencies and a sharper transitional edge, having a shorter transition time, will contain higher frequencies compared to an impulse with a longer transition time. Thus, the method adjusts the transition time to optimize response characteristics of the examination with respect to particular components of interest. Thus, if a component under examination is not particularly responsive to the high frequency components, the transmitted energy can be optimized within the lower frequency band, thereby providing output data that carries more useful information, without increasing the overall level of energy introduced into organs of the subject. Thus, having tested responses with respect to the specified material, the apparatus is programmed at step 302 to achieve the required level of optimization during the examination process, as descried with reference to FIG. 5.

In an alternative embodiment, adjustments of this type can also be made with reference to attributes of an actual person for whom an examination is to be performed upon.

When deployed, manual interaction allows the apparatus to be activated at step 303. A calibration process is then performed at step 304, with the techniques described with reference to FIG. 11 and FIG. 12 being performed without a subject's finger being in place. Thus, characteristics of the device are considered during this calibration process with reference to ambient conditions.

After performing the calibration process, the subject is notified, via the graphical display described with reference to FIG. 2, to the effect that the apparatus is ready. Thus, in response to this notification, a test is performed, as described with reference to FIG. 1, at step 306.

At step 307, test results are transmitted from the apparatus to the mobile telephone itself. In an embodiment, a physical communication channel could be provided between the apparatus and the mobile telephone. However, in the example described with reference to FIG. 1, the examining apparatus physically supports the mobile telephone in a fashion substantially similar to known mobile telephone cases. No actual modification is required to the mobile telephone, except for the downloading of the relevant application. Communication is then performed wirelessly, possibly in accordance with known Bluetooth® protocols. The detecting device is therefore wirelessly paired with the mobile telephone and no further mechanical modifications are required. Thus, having transmitted results at step 307, data is displayed as described with reference to FIG. 2.

In an alternative embodiment, the detecting functionality is included within a mobile telephone itself. This could rely on the existing processing capabilities or additional hardware could be included, possibly comprising an application specific integrated circuit.

At step 308 a question is asked as to whether another test is to be performed and when answered in the affirmative, the calibration process is repeated at step 304. When answered in the negative, the system is de-energized at step 309 to conserve battery power.

FIG. 4

A schematic representation of an examination apparatus embodying the present invention is shown in FIG. 4. A multiplexing environment 401 includes a dielectric membrane supporting at least one group of parallel electrodes. Environment 401 also includes a de-multiplexer for de-multiplexing energizing input pulses, along with a multiplexer for combining selected output signals.

A processor 402 addresses the de-multiplexer and the multiplexer to ensure that the same electrode cannot be both energized as a transmitter and monitored as a receiver during the same coupling operation. An energizing circuit 403 is energized by a power supply 404.

An output from the multiplexing environment 401 is supplied to an output circuit 409 over a first analog line 410. A conditioning operation is performed by the output circuit 409, allowing analog output signals to be supplied to the processor 402 via a second analog line 411. The processor 402 also communicates with a data-communication circuit 412 to facilitate communication with the attached mobile device using a wireless protocol, such as Bluetooth®.

During scanning operations, the processor 402 supplies addresses over address buses 414 to the multiplexing environment 401, to define a pair of capacitively coupled electrodes. An energization operation is performed by applying an energizing voltage, monitoring a resulting output signal and sampling the output signal multiple times to capture data indicative of a peak value and a rate of decay.

Data processing can be performed by the processor 402, such that the mobile telephone is only required to display the processed data. Alternatively, processing capability within the mobile telephone can be used to assist with this data processing exercise. Additional processing could be supported by a local computer system or processing capability could be provided by a cloud-based system.

The generation of an input impulse signal is controlled by the processor 402 by the generation of a control signal on a control line 406. The transition time required has been pre-programmed into the processor 402. This information is conveyed to the energizing circuit via a control bus 408.

FIG. 5

The energizing circuit 403 is detailed in FIG. 5 and includes an input circuit 501 for supplying an input signal to a transmitter electrode. The analog processing circuit 409 provides an output circuit for receiving an output signal from a receiver electrode, when the transmitter electrode and the receiver electrode are capacitively coupled.

In addition, the energizing circuit 403 includes a variable resistance circuit 502 connected in series between the input circuit 501 and the transmitter electrode selected within the multiplexing environment 401. The input circuit generates an input signal in the form of an impulse signal with an initial edge having a transition time. This transition time is adjusted in response to varying the resistance presented by the variable resistance circuit 502.

In the embodiment of FIG. 5, the variable resistance circuit comprises resistors that may be selectively connected between the input circuit and the transmitter electrode. Other types of variable resistance could be provided. For example, digital potentiometers could be deployed. However, in the embodiment of FIG. 5, the resistors are passive elements configured to modify the overall series resistance presented within the circuit.

In the example of FIG. 5, four resistances are present, connected in parallel, identified as a direct connection 510, a first resistor 511, a second resistor 512 and a third resistor 513. The inclusion of four resistances is for illustrative purposes only and other embodiments could include more or fewer.

Resistances 510 to 513 are connected to a first multiplexer 521 which selectively connects one of the resistances 510 to 513 in series with the transmitter electrode. To achieve this, the first multiplexer 521 receives an address from the processor 502. Thus, the processor 502 is programmed with address data, following the testing procedure 302 described with reference to FIG. 3. This information is conveyed to the first multiplexer 521 over the control bus 408.

A selection of direct connection 510 by the first multiplexer 521 does not introduce additional resistance into the circuit. In an embodiment, the level of resistance introduced into the circuit increases by the selection of the first resistor 511, followed by the selection of the second resistor 512, followed by the selection of the third resistor 513. In an embodiment, the increasing resistance may occur arithmetically such that, for example, the first resistor 511 may introduce one ohm, the second resistor 512 may introduce two ohms, and the third resistor 513 may introduce three ohms. Alternatively, the resistances may increase geometrically, such that the first resistor 511 may introduce one ohm, the second resistor 512 may introduce two ohms and the third resistor 513 may introduce four ohms. In this embodiment, the level of introduced resistance is relatively modest, possibly falling within a range of zero-point five ohms to eight ohms. In other embodiments, higher levels of resistance may be appropriate.

The input circuit 501 includes an integrated-circuit-implemented switch 522. This may take the form of an MAX4729 low voltage CMOS analog switch produced by Maxim Integrated of San Jose Calif. However, other devices are available that achieve similar results. Upon activation, the power supply 404 applies a supply voltage 523, typically ranging from two volts to five volts. Activation is achieved in response to receiving an activation signal from the processor 402 on the control line 406.

FIG. 6

Examples of impulse signals providing energizing signals are illustrated in FIG. 6. In this example, the transition is a rising edge, rising from zero volts to the supply voltage which, in an embodiment, may be three point three volts.

A first response 600 is achieved when the direct connection 510 is selected by the first multiplexer 521. Similarly, upon selecting the first resistor 511, a second response 601 is achieved. Again, upon selection of the second resistor 512, a third response 602 is achieved and upon selection of the third resistor 513, a fourth response 603 is achieved.

Each response 600 to 603 has a different transition time which, typically, would be measured in nanoseconds. Thus, as shown in FIG. 6, the first response 600 has a first transition time 610, the second response 601 has a second transition time 611, the third response 602 has a third transition time 612 and the fourth response 603 has a fourth transition time 613.

The actual transition times can vary depending upon specific applications and, as previously stated, more or fewer responses may be available for selection. In an embodiment, the first transition time 610 may be one nano second, the second transition time 611 may be three nano seconds, the third transition time 612 may be five nanoseconds, and the fourth transition time 613 may eight nanoseconds.

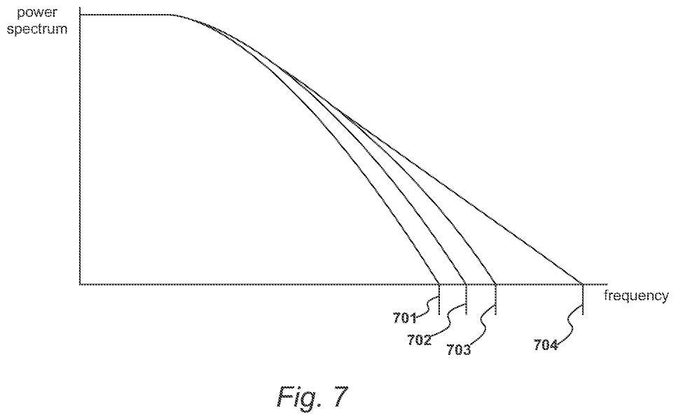

FIG. 7

It is possible to analyze the frequency components of the impulse signals provided as energizing signals. A representation of this is shown in FIG. 7, in the form of a power spectrum. In this example, frequency components up to a frequency of 701 are present for the fourth response 603. Frequency components up to 702 are present for the third response 602. Frequency components up to 703 are present for the second response 601 and frequency components up to 704 are present for the first response 600. In an example, frequency components are measured up to a frequency of 1 GHz (one giga-hertz).

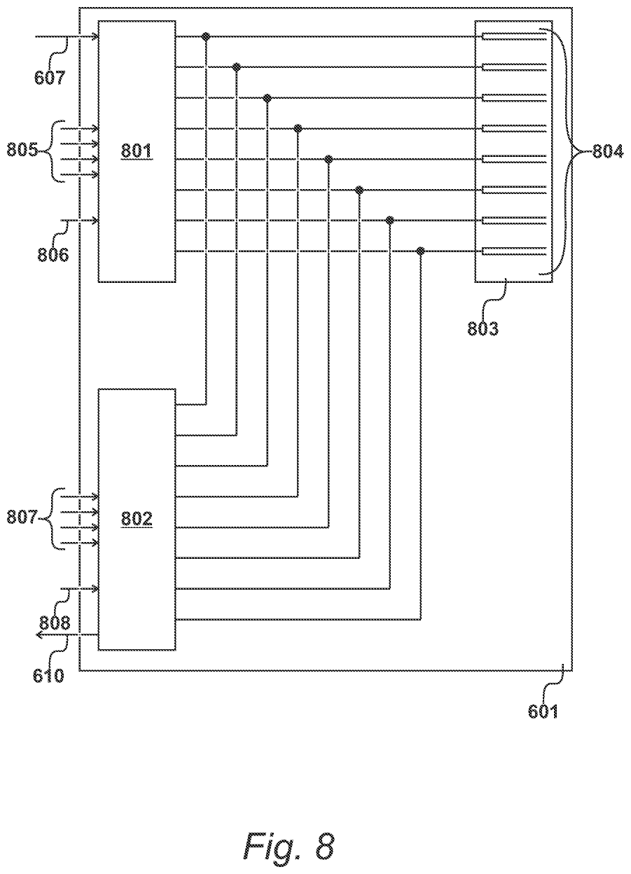

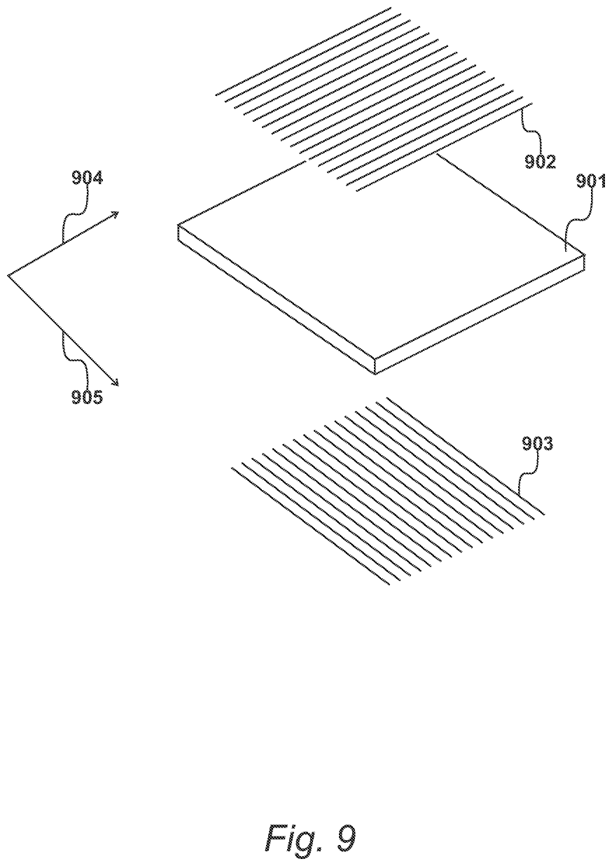

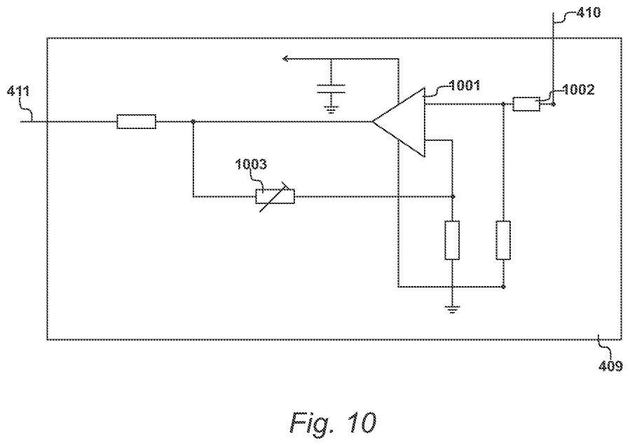

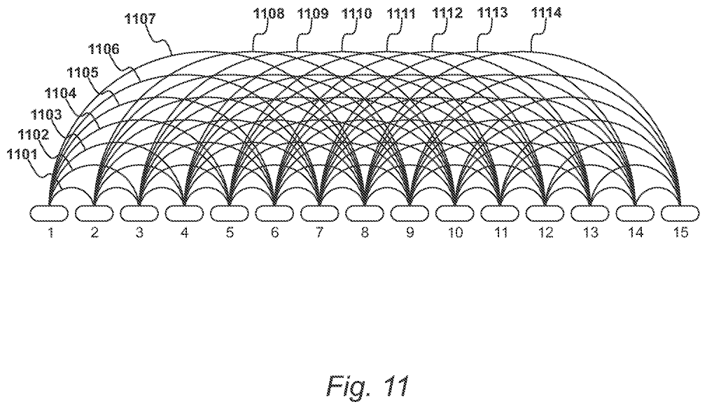

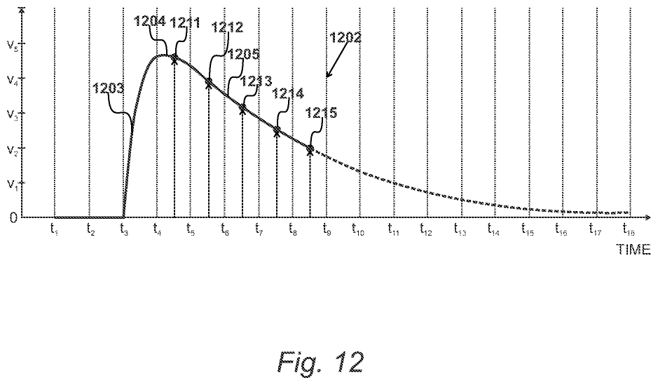

FIG. 8

An example of a multiplexing environment is shown in FIG. 8, in which a second multiplexer 801 supplies input signals from the first multiplexer 521 to a selected transmitter electrode, while a third multiplexer 802 monitors output signals from a selected receiver electrode. A dielectric membrane 803 supports plural parallel electrodes coated with an insulating material to allow them to be brought into contact with a subject's finger. Eight linear electrodes 804 are shown for illustrative purposes but more or fewer electrodes may be included.

The second multiplexer 801 includes a second address bus 805 and an enabling line 806. Similarly, the third multiplexer 802 includes a third address bus 807 and a third enabling line 808. During each electrode coupling operation, addresses are supplied to the second and third address busses but line selection does not occur until respective enabling signals have been received on the respective enabling lines.

FIG. 9

The provision of two mutually orthogonal electrode groups is illustrated in FIG. 9, upon a dielectric membrane 901. The subassembly includes a first group of electrodes 902, along with a second group of electrodes 903. In this example, fifteen electrodes are provided in each group and for each group, the electrodes are substantially linear and substantially parallel. However, the groups are mutually offset and, in this embodiment, arranged in mutually orthogonal orientations.

The first group of electrodes 901 are aligned in an x dimension, illustrated by a first arrow 904 and the second group of electrodes 903 are aligned in a y dimension, as illustrated by a second arrow 905. Layering is achieved by coupling electrodes of a first set (selected from a group) and then repeating a scanning operation by coupling electrodes in a second set, selected from the same group. Thus, layering operations performed by the first group of electrodes 902 achieve a layering operation in the direction of the second arrow 905. Similarly, the second group of electrodes 903 achieve a similar layering operation in the direction of the first arrow 904.

FIG. 10

An example of output circuit 409 is shown in FIG. 10. Signals received on the first analog line 410 are supplied to a buffering amplifier 1001. During an initial set-up procedure, a variable feedback resistor 1003 is trimmed to optimize the level of monitored signals supplied to the processor 402 via the second monitoring line 411.

FIG. 11

The electrodes of the first group 902 are shown in FIG. 11, numbered 1 to 15. Input signals are generated for application to any of the electrodes 1 to 15 as a transmitter electrode. In addition, output signals may be monitored from any remaining one of the electrodes in the group as a receiver electrode, wherein a peak value of an output signal is substantially indicative of permittivity and a decay rate of an output signal is substantially indicative of conductively. Thus, during each operation, an energized transmitter electrode and a monitored receiver electrode define a capacitively coupled electrode pair.

From the available electrodes 1 to 15 of the selected group, a first set of n electrodes is selected. Thus, in the example shown in FIG. 11, all fifteen electrodes are selected as the first set of n electrodes. However, it is not necessary for all of the available electrodes to be selected in this way, such that, in alternative embodiments, the selected set may contain fewer electrodes than the total number in the group.

Capacitively coupled electrode pairs are established, in which each of the first set of n electrodes is capacitively coupled with a second set of m electrodes selected from the first set of n electrodes. Thus, the second set of m electrodes is a subset of the first set of n electrodes. Thus, for each selected electrode of the first set, a respective second set of m electrodes is identified. These m electrodes are sequentially capacitively coupled with the selected electrode of the first set.

In an embodiment, this capacitive coupling may occur in parallel, requiring multiple analog to digital conversion devices operating in parallel. However, in this embodiment, capacitive coupling occurs sequentially such that, at any instant, only one electrode of the first set is coupled with only one electrode of the second set. To achieve this coupling, either electrode may be energized as a transmitter, with the other electrode of the pair being monitored as the receiver.

Furthermore, in this embodiment, each second set of m electrodes are the nearest neighbouring electrodes to an electrode selected from the first set of n electrodes. Consequently, the number of electrodes present in the second set of m electrodes represents a degree of layering.

Following this method, all of the resulting electric fields are illustrated in FIG. 11. The first set of n electrodes consists of all fifteen available electrodes within the group. The degree of layering is seven, as illustrated by electric fields 1101 to 1107. Each of the first set of n electrodes is capacitively coupled with a second set of m electrodes. Thus, when considering the first electrode 1 as being a member of the first set, it is capacitively coupled with electrodes 2 to 8 of the second set. Electrode 1 is not capacitively coupled with electrodes 9 to 15. Thus, the second set of m electrodes (2 to 8) with reference to the first electrode 1, are the nearest neighbouring electrodes to electrode 1, selected from the remaining n electrodes of the first set. Thus, having achieved a seventh degree of layering, it would be necessary to couple electrode 1 with electrode 9, should an eighth degree of layering be required.

The selected electrodes may be capacitively coupled in any order, provided that all of the electric fields illustrated in FIG. 11 are realised. Investigations have identified advantages with respect to having multiple couplings with the end electrodes which, in this example, are identified as electrode 1 and electrode 15. Such an approach provides useful layering data. However, in the present embodiment, many more layering opportunities are established through a dynamic process.

As previously described, data derived from the seventh degree of layering is achieved by electric field 1107, from the coupling of electrode 1 with electrode 8. Similar seventh degree fields are shown at 1108 (coupling electrodes 2 and 9), 1109 (coupling electrodes 3 and 10), 1110 (coupling electrodes 4 and 11), 1111 (coupling electrodes 5 and 12), 1112 (coupling electrodes 6 and 13), 1113 coupling electrodes 7 and 14) and 1114 (coupling electrodes 8 and 15).

FIG. 12

A monitored output signal 1202 has been amplified by the circuit described with reference to FIG. 10 and is then sampled by an analog to digital convertor forming part of the processor 402. The monitored output signal 1202 has a rising edge 1203, a peak 1204 and a falling edge 1205. The rising edge 1203 up to the peak level 1204 will be determined predominantly by permittivity characteristics. Similarly, the falling edge 1205 represents a decay of the induced field and the rate of decay will be determined predominantly by conductivity characteristics. Thus, by recording multiple samples, it is possible to obtain rich coupling data sets. To achieve this, as illustrated in FIG. 12, a first sampling point 1211 is followed by a second sampling point 1212, followed by a third sampling point 1215, a fourth sampling point 1214 and a fifth sampling point 1215.

The processor 402 is responsible for initiating the generation of an input signal, therefore the processor is instructed with an appropriate delay period before initiating the sampling process. This delay is determined empirically and aims to place the first sampling point at the peak value. However, a degree of tolerance is permitted, as illustrated in FIG. 12, given that the same delay period is used for each coupling operation, allowing comparisons to be made between similar examples. However, for the purposes of this embodiment, it should be appreciated that each coupling operation results in the generation of an output signal substantially similar to that shown at 1202, which in turn generates a coupling data set containing five data points. In other embodiments, more or fewer data samples may be recorded.

In an alternative embodiment, the sampling and processing capabilities of the processor 402 are enhanced. For example, an embodiment may take up to twelve samples while approaching the peak, followed by a further two-hundred-and-fifty samples after the peak.

The invention claimed is:

1. An apparatus for examining objects, comprising:
at least one transmitter electrode and at least one receiver electrode;
an input circuit configured to supply an input signal to said at least one transmitter electrode;
an output circuit arranged to receive an output signal from said at least one receiver electrode, wherein said at least one transmitter electrode and said at least one receiver electrode are capacitively coupled;
a variable resistance circuit connected in series between said input circuit and said at least one transmitter electrode, wherein:
said input signal is an impulse signal with an initial edge having a transition time; and
said transition time is adjustable in response to varying a resistance presented by said variable resistance circuit.

2. The apparatus of claim 1, wherein said variable resistance circuit comprises a plurality of resistors that are selectively connected between said input circuit and said at least one transmitter electrode.

3. The apparatus of claim 2, further comprising a first multiplexing device for selecting one of said plurality of resistors.

4. The apparatus of claim 1, wherein said input circuit comprises an integrated circuit implemented switch.

5. The apparatus of claim 1, wherein said at least one transmitter electrode and said at least one receiver electrode are selected from a first set of substantially parallel electrodes.

6. The apparatus of claim 5, further comprising:
a second multiplexing device for supplying an input signal to any one of said first set of substantially parallel electrodes; and
a third multiplexing device for receiving an output signal from any one of said first set of substantially parallel electrodes.

7. The apparatus of claim 5, further comprising a second set of substantially parallel electrodes, wherein said second set of substantially parallel electrodes is orthogonally displaced with respect to said first set of substantially parallel electrodes.

8. The apparatus of claim 5, further comprising a housing to facilitate an interaction with a finger upon said first set of substantially parallel electrodes to facilitate a measurement of blood glucose concentrations.

9. The apparatus of claim 8, wherein:
said housing forms a case for a mobile communication device;
the apparatus further comprises a processor for initiating generation of input impulses and sampling processed output signals from said output circuit; and
data from said processor is supplied wirelessly to said mobile communication device.

10. The apparatus of claim 5, further comprising an infra-red measuring device for assessing a temperature of a deployed finger.

11. A method of examining objects, comprising the steps of:
passing an electric field through an object, in response to energizing a transmitter electrode; and
monitoring a receiver electrode, wherein:
an energizing signal is an impulse signal with an initial edge having a transition time; and
said transition time is adjusted to optimize response characteristics of an examination with respect to particular components of interest.

12. The method of claim 11, wherein said transition time is adjusted by adjusting a resistance of a transmission path to said transmitter electrode.

13. The method of claim 12, wherein said resistance is adjusted by selecting a series connected resistor from a plurality of available resistors.

14. The method of claim 13, wherein said selecting step is performed by a multiplexer.

15. The method of claim 11, wherein said impulse signal is generated by an integrated low voltage switch.
